(19) United States
(12) Patent Application Publication
(10) Pub. No.: US 2003/0211465 A1
(43) Pub. Date: Nov. 13, 2003

(54) Immunokine composition and method

(76) Inventors: David D. Mundschenk, Dania, FL (US); Paul F. Reid, Hollywood, FL (US)

Correspondence Address: INTELLECTUAL PROPERTY GROUP, FREDRIKSON & BYRON, P.A., 4000 PILLSBURY CENTER, 200 SOUTH SIXTH STREET, MINNEAPOLIS, MN, 55402, US

(21) Appl. No.: 10/292,164

(22) Filed: Nov. 12, 2002

Related U.S. Application Data
(62) Division of application No. 09/533,454, filed on Mar. 23, 2000.
Application No. 09/533,454 is a continuation-in-part of application No. 09/368,834, filed on Aug. 5, 1999.
Application No. 09/368,834 is a continuation of application No. 08/908,212, filed on Aug. 7, 1997, now Pat. No. 5,989,857.
Application No. 08/908,212 is a continuation of application No. 08/644,399, filed on May 10, 1996, now abandoned.

(51) Int. Cl. C12Q 1/70; A61K 38/19; C07K 14/52

(52) U.S. Cl. 435/5; 424/85.1; 530/351

(57) ABSTRACT

A composition and method for preventing HIV infection of mammalian cells. One aspect of the invention relates to an anti-immunodeficiency virus immunokine capable of binding to a cellular protein in a manner that prevents HIV infection of that cell. The compositions can include either an active bioactive polypeptide, such as native cobratoxin, and/or an inactivated bioactive polypeptide, such as cobratoxin in which one or more of the native disulfide bridges have been prevented from forming. The term “immunokine” is used to refer to an inactivated bioactive polypeptide, whether inactivated by chemical, genetic, and/or synthetic means as described herein, with the proviso that a corresponding active bioactive polypeptides can be included where applicable (e.g., for in vitro use).

### CROSS REFERENCE TO RELATED APPLICATIONS

This application is a divisional of prior application Ser. No. 09/533,454, filed on Mar. 23, 2000, which is a continuation-in-Part of prior application Ser. No. 09/368,834, filed on Aug. 5, 1999, which is a continuation of prior application Ser. No. 08/908,212, filed on Aug. 7, 1997, now U.S. Pat. No. 5,989,857, which is a continuation of U.S. patent application filed May 10, 1996 and assigned Ser. No. 08/644,399 for POLYPEPTIDE COMPOSITIONS AND METHODS, the entire disclosure of which is incorporated herein by reference.

### TECHNICAL FIELD

The present invention relates to the treatment and prevention of viral infections, including HIV infections.

### BACKGROUND OF THE INVENTION

The lack of an effective vaccine and the increase in antiretroviral drug treatment failures has led the HIV research community to continue the search for novel approaches to treat HIV infection. HIV can be inhibited at a number of different steps in its lifecycle within the cell or, alternatively, vaccines and immune based therapies can eliminate HIV-infected cells directly.

The HIV lifecycle involves binding of the virus to specific cell receptors. These receptors include CD4 and the recently discovered co-receptors called chemokine receptors. Following receptor binding the virus is internalized into the cell and the viral RNA is converted into DNA by a process called reverse transcription. Reverse transcription requires an enzyme called reverse transcriptase, a common target for antiretroviral drugs. Following reverse transcription, the viral DNA is transported to the nucleus of the cell where it integrates into the host's chromosome by way of a process called integration; a process that requires the enzyme integrase. Following integration into the host chromosome, the integrated DNA serves as a template for transcription of viral gene products required for replication or for packaging into new progeny virus. These viral mRNAs code for enzymatic or structural proteins, some of which require cleavage by specific proteases to produce infectious viral particles. The new, much publicized HIV drugs called protease inhibitors, inhibit this cleavage step resulting in the production of non-infectious viral particles.

In turn, anti-HIV compounds have been directed against HIV entry (entry inhibitors), HIV fusion (fusion inhibitors), reverse transcription (nucleoside and non-nucleoside reverse transcriptase inhibitors), HIV integration (integrase inhibitors), HIV transcription inhibitors, and the aforementioned protease inhibitors. Inhibition of HIV at these different sites results in a specific pattern of HIV gene expression that requires sophisticated molecular techniques to decipher.

For instance, the (NIAID) categorizes anti-HIV compounds as having either viral targets or cellular targets. Examples of those having viral targets include Gag proteins and precursors (e.g., capsid structural protein, matrix protein, RNA binding protein, and other Gag proteins,); viral enzymes (e.g., polymerase, protease and integrase); envelope proteins (e.g., surface glycoprotein and transmembrane glycoprotein); accessory and regulatory proteins (e.g., Tat, Rev, Nef, Vif, Vpr, Vpx and Tev); and nucleic acids (e.g., HIV RNA).

Examples of anti-HIV compounds having cellular targets include cellular receptors such as the immunoglobulin superfamily (e.g., CD4); and chemokines (seven-transmembrane) receptor superfamily, examples of which include CXCR4 (also known as fusin, LESTR, NPY3R), and CCR5 (also known as CKR-5, CMKRB5).

Chemokines are a large family of low molecular weight, inducible, secreted, proinflammitory cytokines which are produced by various cell types. See, for instance, Au-Yuong, et al., U.S. Pat. No. 5,955,303, which describes the manner in which chemokines have been divided into several subfamilies on the basis of the positions of their conserved cysteines. The CXC family includes interleukin-8 (IL-8), growth regulatory gene, neutrophil-activating peptide-2, and platelet factor 4 (PF-4). Although IL-8 and PF-4 are both polymorphonuclear chemoattractants, angiogenesis is stimulated by IL-8 and inhibited by PF-4. The CC family includes monocyte chemoattractant protein-1 (MCP-1), RANTES (regulated on activation, normal T cell-expressed and secreted), macrophage inflammatory proteins (MIP-1.alpha., MIP-1.beta.), and eotaxin. MCP-1 is secreted by numerous cell types including endothelial, epithelial, and hematopoietic cells, and is a chemoattractant for monocytes and CD45RO+lymphocytes (Proost, P. (1996) Int J. Clin. Lab. Res. 26: 211-223; Raport, C. J. (1996) J. Biol. Chem. 271: 17161-17166).

Cells respond to chemokines through G-protein-coupled receptors. These receptors are seven transmembrane molecules which transduce their signal through heterotrimeric GTP-binding proteins. Stimulation of the GTP-binding protein complex by activated receptor leads to the exchange of guanosine diphosphate for guanosine triphosphate and regulates the activity of effector molecules. There are distinct classes of each of the subunits which differ in activity and specificity and can elicit inhibitory or stimulatory responses.

Chemokine receptors play a major role in the mobilization and activation of cells of the immune system. The effects of receptor stimulation are dependent on the cell type and include chemotaxis, proliferation, differentiation, and production of cytokines. Chemokine stimulation produces changes in vascular endothelium, chemotaxis to sites of inflammation, and activates the effector functions of cells (Taub, D. D. (1996) Cytokine Growth Factor Rev. 7: 355-376).

The chemokine receptors display a range of sequence diversity and ligand promiscuity. The known chemokine receptor protein sequence identities range from 22 to 40%, and certain receptors can respond to multiple ligands. Although mainly expressed in immune cells, viral homologues are expressed by human cytomegalovirus and Herpesvirus saimiri. The chemokine receptor known as the Duffy blood group antigen binds both CC and CXC family chemokines and serves as the receptor on erythrocytes for the malarial parasite Plasmodium vivax. Members of the chemokine receptor family are used as co-receptors with CD4 for HIV-1 entry into target cells. Several receptors have recently been cloned.

See also, U.S. Pat. No. 5,994,515 (Hoxie) which describes the manner in which the human immunodeficiency viruses HIV-1 and HIV-2 and the closely related simian immunodeficiency viruses (SIV), all use the CD4 molecule as a receptor during infection. Other cellular molecules have long been suspected to form an essential component of the cellular HIV-1 receptor; however, the nature of such cellular molecules was not known until the discovery of fusin (Feng et al., 1996, Science 272:872-876).

Recently, two molecules, fusin, which is now known as CXCR4 (also known as Lestr, LCR-1, and HUMSTR) and CCR5, which are members of the chemokine receptor family of proteins, have been shown to function with CD4 as coreceptors for HIV-1 isolates that are tropic for T-cell lines or macrophages, respectively). Results to date indicate that the use of chemokine receptors is a general property of all human and nonhuman lentiviruses.

CXCR4 is a cellular protein which in conjunction with CD4, forms a functional cellular receptor for entry of certain strains of HIV into cells. This protein is a member of a family of molecules that bind chemokines which are involved in the trafficking of T cells and phagocytic cells to areas of inflammation (Power and Wells, 1996, Trends Pharmacol. Sci. 17:209-213).

CXCR4 fulfills the requirements of an HIV receptor co-factor. It renders a number of murine, feline, simian, quail, and hamster cell lines, as well as human cell lines, which cells are normally resistant to HIV-1 entry, fully permissive for HIV-1 env mediated syncytia formation. In addition, the T cell tropic HIV strain HIV-1 IIIB, is capable of infecting both murine and feline cells which co-express human CD4 and CXCR4. However, the macrophage tropic strain Ba-L, is not capable of infecting cells which co-express both CXCR4 and CD4. These results suggest that CXCR4 can serve as a co-factor for T-tropic, but not M-tropic, HIV-1 strains (Feng et al., 1996, supra). Moreover, the finding that change from M to T-tropic viruses over time in infected individuals correlates with disease progression suggests that the ability of the viral envelope to interact with CXCR4 represents an important feature in the pathogenesis of immunodeficiency and the development of full blown AIDS.

Current anti-HIV therapy includes the use of compounds which inhibit various aspects of HIV replication in a cell such as inhibition of replication and/or transcription of viral nucleic acid and inhibition of protein processing. While these therapies, particularly when used in combination with one another, are effective, they are frequently short-lived in that viral strains rapidly develop that are resistant to one or more of the compounds used. There therefore remains an acute need to develop additional therapies and strategies for preventing HIV infection in humans.

On a separate subject, a previous patent issued to the present assignee, U.S. Pat. No. 5,989,857, describes, inter alia, a method of preparing a bioactive polypeptide in a stable, inactivated form, the method comprising the step of treating the polypeptide with ozonated water in order to oxidize and/or stabilize the cysteine residues, and in turn, prevent the formation of disulfide bridges necessary for bioactivity. The method can involve the use of ozonated water to both oxidize the disulfide bridges in a bioactive polypeptide, and to then stabilize the resultant cysteine residues. Optionally, and preferably, the method can involve the use of ozonated water to stabilize the cysteine residues, and thereby prevent the formation of disulfide bridges, in a polypeptide produced by recombinant means in a manner that allows the polypeptide to be recovered with the disulfide bridges unformed.

What are clearly needed are improved methods and compositions for the treatment and prevention of HIV.

### SUMMARY OF THE INVENTION

The present invention provides a composition and method for preventing HIV infection of mammalian cells. One aspect of the invention relates to an anti-immunodeficiency virus immunokine capable of binding to a cellular protein in a manner that prevents HIV infection of that cell. In another aspect, the immunodeficiency virus is selected from the group consisting of HIV-1,HIV-2 and SIV. In another aspect, the invention relates to the identification of a biologic anticholinergic agent capable of binding to a cellular protein in a manner that prevents HIV infection of that cell. In yet another aspect the invention relates to an anti-immunodeficiency virus immunokine derived from a biologic anticholinergic agent which can be administered in vivo for the treatment of HIV infection. The immunodeficiency virus can be selected from the group consisting of Lentiviruses (HIV-1, HIV-2, SIV, EIAV, BIV, FIV and FeLV).

Compositions of this invention can include either an “active bioactive polypeptide”, such as native cobratoxin, and/or an “inactivated bioactive polypeptide”, such as cobratoxin in which one or more of the native disulfide bridges have been prevented from forming. While not presently preferred for in vivo applications, it appears that the active polypeptides exhibit the desired antiviral activity, and in turn, can be used for in vitro (e.g., diagnostic) applications. The term “immunokine” will generally be used to refer to an inactivated bioactive polypeptide, whether inactivated by chemical, genetic, and/or synthetic means as described herein, with the proviso that a corresponding active bioactive polypeptides can be included where applicable (e.g., for in vitro use).

A composition of this invention is useful in preventing infection of a cell, both with in terms of treating existing HIV spread within an infected individual as well as preventing initial HIV infection of that individual. As such, the composition can be useful in limiting the spread of virus from one cell to another in an infected host and, if present, (i.e. circulating within a host) prior to exposure (but not productive infection) of a cell.

Proteins such as those from venoms, as described herein, have long been recognized for their ability to bind to specific receptors on the surface of human cells. These neurospecific proteins bind to such common receptors as the acetylcholine receptor for example. Significantly less well known than the interactions between venom proteins and human cells is the ability of these venoms to cause cells to migrate toward or in response to the venom proteins. This cellular activity is called chemotaxis and, until the characterization of these venom proteins by the present Applicants, this property has only been attributed to compounds called chemokines produced in immune cells. For these reasons, we will heretofore refer to our venom proteins as “immunokines”.

In yet another aspect of the invention, the protein to which the immunokine of the invention binds is one or more of a chemokine receptor protein, preferably, an HIV receptor protein and/or a cellular cofactor for a cellular HIV receptor protein. More preferably, the protein to which the immunokine of the invention binds is selected from the group consisting of CD4, CXCR4 and CCR5; and most preferably, the protein to which the immunokine binds is CD4/CXCR4 and/or CD4/CCR % complexes.

In another aspect of the invention, the immunokine is most preferably selected from the group consisting of post-synaptic alpha-neurotoxins (Group II) and anticholinergic peptides.

The invention also relates to an isolated DNA encoding an immunokine capable of binding to a cellular protein in the manner described herein.

The invention also relates to a method of inhibiting infection of a cell by HIV comprising adding to the cell an anti-immunodeficiency virus immunokine capable of binding to a cellular protein on the cell, wherein upon binding of the immunokine to the cellular protein infection of the cell by HIV is inhibited.

Also included in the invention is a method of treating HIV infection in a human comprising administering to the human an anti-immunodeficiency virus immunokine capable of binding to a cellular protein on a cell, wherein upon binding of the immunokine to the cellular protein, infection of the cell by HIV is inhibited, thereby treating the HIV infection in the human.

The invention further includes a method of obtaining an anti-immunodeficiency virus immunokine capable of binding to a cellular protein on a cell, in one embodiment the method comprising an oxidative process for the chemical production of immunokine by combining ozone with the protein of interest, e.g., a native or synthetic neurotoxin.

Also included in the invention is a method of identifying a target cell for immunodeficiency virus infection, the method comprising adding to a population of cells native or synthetic active bioactive polypeptide (e.g., alpha-cobratoxin) or an anti-immunodeficiency virus immunokine capable of binding to a cellular protein on a cell, wherein binding of the immunokine to a cell in the population is an indication that the cell is an immunodeficiency virus target cell.

In addition, there is provided a method of identifying a candidate anti-immunodeficiency virus compound. This method comprises isolating a test compound capable of binding to an active bioactive polypeptide such as alpha-cobratoxin or an anti-immunodeficiency virus immunokine, which immunokine binds to a cellular protein, and assessing the ability of the test compound to inhibit infection of a cell by an immunodeficiency virus in an antiviral assay, wherein inhibition of infection of the cell by the immunodeficiency virus in the presence of the test compound is an indication that the test compound is an anti-immunodeficiency virus compound.

### DETAILED DESCRIPTION

In one preferred embodiment, the invention relates to an antiviral, anticholinergic protein and immunokine which binds to one or more cellular proteins essential for entry of a virus into a cell expressing that protein The immunokine of the invention is an antiviral immunokine in that it is an immunokine which binds to one or more cellular proteins that are essential for virus entry into the cell in which the cellular protein is expressed. By binding to the cellular protein, the immunokine of the invention inhibits entry of the virus into the cell and is therefore termed an antiviral immunokine despite the fact that it does not bind to a viral protein, but rather, binds to a cellular protein. The invention further relates to an antiviral immunokine which binds to one or more cellular proteins essential for entry of a virus into a cell expressing that protein.

The virus against which the antiviral immunokine is directed is an immunodeficiency virus, that is, a virus which causes an immunodeficiency disease. Thus, the immunokine of the invention is termed an anti-immunodeficiency virus immunokine. Such immunodeficiency virus should be construed to include any strain of HIV or SIV, as well as other lentiviruses (FIV, FeLV, BIV, and EIAV).

By “HIV” as used herein, is meant any strain of a human immunodeficiency virus belonging to the group of either HIV type 1 or HIV type 2. By “SIV” as used herein is meant any of five recognized strains of SIV (SIVmac, SIVsmm, SIVagm, SIVmnd and SIVcpz) which are known to infect non-human primates.

Without intending to be bound by theory, it appears that both native alpha-cobratoxin and an immunokine of the invention are each capable of binding to a cellular protein required to form a functional cellular receptor for entry of HIV into a cell. In one preferred embodiment, the immunokine of the invention is an immunokine which binds to a cellular receptor and/or to a cellular co-factor required for entry of HIV into a cell. A “cellular co-factor” as used herein, is defined as a protein which is required, in association with a cellular receptor for HIV, for entry of HIV into cells.

According to the invention, the polypeptides (e.g., native or immunokine) of the invention is useful in a method of inhibiting infection of a cell by HIV as described herein. Moreover, the immunokine of the invention is useful in a method of screening compounds for anti-HIV activity as described herein. Additional uses for alpha-cobratoxin or an immunokine of the invention include the identification of cells in the body which are potential targets for viral infection. The immunokine is thus also useful for the isolation of such cells using flow cytometry technology or other cellular isolation techniques which are common in the art. The invention also relates to methods of use of the immunokine of the invention, which methods include diagnostic and therapeutic uses.

By “antiviral activity” as used herein, is meant an immunokine which when added to an immunodeficiency virus or to a cell to be infected with such a virus, mediates a reduction in the ability of the virus to infect and/or replicate in the cell compared with the ability of virus to infect and/or replicate in the cell in the absence of the immunokine. Examples of assays for antiviral activity are described in detail in the experimental detail section and include, but are not limited to, reverse transcriptase assays, immunofluorescence assays, assays for formation of syncytia, antigen capture assays and the like.

Immunokine Preparation

A composition of this invention can be prepared in any suitable manner. For instance, native cobratoxin can be obtained and used in its native (e.g., unmodified) form, and is shown to inhibit HIV infection of cells (PMNC) with a similar efficacy to the corresponding alpha-immunokine described herein. Toxins themselves can be chemically modified (e.g., using ozone, performic acid, iodoacetamide etc.), and other cobratoxin homologues (see Group II) can be prepared. Toxin modifications include site-directed mutants (mono and poly-substituted mutants such as tryptophan, tyrosine, lysine and arginine), chimeras and other homologous peptide fragments produced from the parent protein through genetic engineering or synthetic peptide production.

An inactivated bioactive polypeptide (e.g., immunokine) of this invention can be prepared using any suitable means. As described herein, the immunokine can be chemically produced in an oxidative process in combination with the protein of interest, e.g., a neurotoxin. The use of ozone treatment to prepare the immunokine is particularly preferred, e.g., in view of the simplicity of manufacture, the modest facility requirements and self sterilizing nature of the production procedure. Under controlled conditions, ozone specifically modifies certain amino-acids such as methionine, cysteine and tryptophan to methionine sulphone, cysteic acid and kynurenine respectively. Cobratoxin has no methionine, ten (10) cysteine and one (1) tryptophan residues.

Other procedures can be used as well, though these with each such procedure providing a product that varies in its relative potencies when compared to immunokine produced with ozone. Those procedures include the use of hydrogen peroxide, performic acid, carboxyamidomethylation, iodoacetamide, iodoacetic acid and Oxone (Caro's Acid) but includes any chemical agent that acts as an oxidizer or alkylator that can render proteins like cobratoxin atoxic and suitable for administration to a host. The circumstances where a difference procedure would be employed would be if the resultant product demonstrated better therapeutic activity in other applications, for example superior immuno-modulatory, anti-tumor or anti-viral activity, but they emphasize the importance of breaking the disulphide bonds with a concomitant conformational reorganization similar to that during disulphide oxidation. The requirement for scission of all the disulphide bonds for optimal function has not yet been fully investigated but sufficient bonds must be broken to render a protein like alpha-cobratoxin safe for administration to a host.

Applicant's parent application (now U.S. Pat. No. 5,989,857) described, inter alia, a method that involved bubbling ozone through a solution (10 mg/ml) of cobratoxin in water. This approach could be used, for instance to produce 12 gram batches with a concentrate that could be diluted to any desired concentration. This approach typically involved a 6-8 hour process requiring close monitoring to determine the optimal endpoint. The endpoint of the reaction was typically determined by toxicity studies in mice. Ozonation was determined to be complete when mice survived a 1 mg (0.1 cc) injection. It was determined by this technique, however, that excess ozone could adversely effect the quality of the final drug product.

In a presently preferred embodiment (for the ozonation of neurotoxins to make therapeutic molecules) the present invention now uses only enough ozone to render the toxin atoxic (breaking the disulphide bonds) while minimizing damage to other sensitive sites of oxidation. Secondly, it is now preferred to ozonate physiological saline (0.9% NaCl) such that it contains a known, preferred amount of ozone which is then added to solubilized toxin in the 0.9% NaCl. The oxidation is stoichiometic as described below.

Those skilled in the art will be able to determine a stoichiometric approach, given the present description, as exemplified by the use of cobratoxin as follows:

$$\begin{matrix}In theory , 1 ug \text{/} ml of Ozone contains 0.02083 umoles \text{/} ml = \frac{1 ug \text{/} ml Ozone}{48 MW} \\ Likewise , 1 ug of toxin contains 0.0001276 umoles of toxin = \frac{1 ug \text{/} ml toxin}{7831 MW}\end{matrix}$$

If one multiplies by ten to account for the sulphurs (half cystines) to be oxidized by the ozone (i.e., 0.000127 umoles×10) one obtains the value of 0.00127 umoles of sulphur molecules (half cystines).

Experimental models were used to confirm these assumptions. Varying amounts of toxin (25 mg-1230 mg dissolved in 10 ml solution) were brought up to a final volume of 1 liter using ozonated saline, as described above. In this model, samples containing 25 mg/l to 300 mg/l toxin were not toxic in mice while the 610 mg and 1230 mg samples killed mice. Additional experiments used 19.1 ug/ml ozone dissolved in 0.9% saline in which samples containing 600 mg/l and 700 mg/l of toxin were oxidized. The 600 mg/l samples were not toxic in mice and the 700 mg/l samples killed mice, thus defining the range of use.

In one aspect, the present invention provides a method of preparing a parenteral composition comprising an immunokine (e.g., an immunokine), the method comprising the steps of:

a) identifying a polypeptide having a biological activity dependent on the presence of one or more disulfide bridges in its tertiary structure,

b) preparing a cDNA strand encoding the polypeptide,

c) expressing the cDNA under conditions in which the polypeptide is recovered in an inactive form due to the failure to form one or more disulfide bridges, and

d) recovering the inactive polypeptide and formulating it into a composition suitable for parenteral administration to a host.

In another aspect, the invention provides a composition comprising an immunokine that has been rendered inactive by virtue of the failure to form one or more of its disulfide bridges. In a related aspect, the invention provides a composition for in vivo administration comprising a bioactive immunokine that has been inactivated in the manner described herein.

The method can be used to prepare immunokines from, or based upon, a variety of natural compounds, including “Group I neurotoxins” (namely, toxins affecting the presynaptic neurojunction), Group II neurotoxins (namely those affecting the postsynaptic neurojunction), and Group III neurotoxins (those affecting ion channels). cDNA sequences for such polypeptides are generally known, or can be determined using conventional techniques.

The cDNA can be expressed using any suitable expression system, under conditions in which the product can be recovered with one or more disulfide bridges unformed. Suitable expression systems include heterologous host systems such as bacteria, yeast or higher eucaryotic cell lines. Examples of useful systems are described, for instance, in “Foreign Gene Expression in Yeast: a Review”, Romanos, et al., Yeast, 8:423-488 (1992). See also, “Yeast Systems for the Commercial Production of Heterologous Proteins”, Buckholz, et al., Bio/Technology 9:1067-1072 (1991), the disclosures of both Romanos et al. and Buckholz et al. being incorporated herein by reference.

These articles are generally directed at the more common goal of affirmatively achieving posttranslational processing and extracellular secretion. Under such conditions, the formation of appropriate disulfide linkages would be included as a necessary step. Given the present description, however, these articles, and the techniques described therein, will be of considerable use to those skilled in the art in achieving the recovery of the unfolded product, e.g., by intracellular expression in yeast.

Preferably, the cDNA is expressed using a microbial expression system, such as Escherichia coli, Saccharomyces cerevisiae and Pichia pastoris. From a safety and environmental perspective it is preferable that the cDNA is expressed in a microbial expression system under conditions in which the product is cytoplasmically produced, as opposed to extracellularly secreted. In an exemplary embodiment, the immunokine is expressed using a microbial expression system, under conditions in which the leader sequence of naturally-occurring cDNA is removed and replaced with only the initiation codon.

Immunokines of the present invention are generally stable under suitable conditions of storage and use in which the disulfide bonds are prevented from spontaneously reforming, or are allowed to reform in a manner that precludes the undesirable activity of the immunokine. Optionally, and preferably, once the inactive polypeptide has been recovered, it is treated by suitable means to ensure that the cysteine residues do not spontaneously reform to form disulfide bridges. An example of a preferred treatment means is the use of ozone treatment as described herein.

In another optional, and alternative, embodiment a immunokine such as neurotoxin is produced in an inactive form using the Pichia expression system described herein. To the best of Applicants knowledge, the prior art fails to teach or suggest the preparation of a toxin in inactive form by the route of cytoplasmic expression in yeast.

The method and composition of the present invention provide a unique and valuable tool for the synthesis and recovery of bioactive immunokines in a manner capable of diminishing undesirable activity, yet retaining other useful properties of the immunokine (such as immunogenicity and antiviral activity).

As used herein, the following words (and inflections thereof) and terms will have the meanings ascribed to them below:

“bioactive” will refer to a polypeptide capable of eliciting at least one biological response when administered in vivo.

“polypeptide” will refer to any biomolecule that is made up, at least in part, of a chain of amino acid residues linked by peptide bonds.

“inactive” will refer to a polypeptide that is provided in a form in which at least one form of its bioactive responses is substantially terminated or decreased to a desired extent.

“neurotoxin” will refer to a bioactive polypeptide wherein at least one activity (e.g., binding to the acetylcholine receptor) produces a toxic effect on the nervous system of a mammalian host.

The method of the present invention involves an initial step of identifying a bioactive immunokine having a tertiary structure in which bioactivity is dependent, at least in part, on the formation of one or more disulfide bridges between cysteine residues. Typically, the immunokine will be one that is naturally secreted in the course of its synthesis, since it is the secretion process that will provide the necessary posttranslational steps, including disulfide bond formation. Preferably, the immunokine is one that is stable when recovered and that retains other desirable properties in the unfolded state, such as immunogenicity and/or antiviral, anti-tumor or wound healing activity.

The amino acid sequence and tertiary structure of a number of bioactive polypeptides is known. Suitable immunokines include those in which one or more disulfide bridges are known to form in the natural configuration, and in which such bridge(s) are necessary for the bioactivity of the immunokine. Such bridges can be of either an intramolecular (i.e., within a single polypeptide) nature and/or an intermolecular (e.g., between discrete subunits) nature.

Secreted or cell-surface proteins often form additional covalent intrachain bonds. For example, the formation of disulfide bonds between the two —SH groups of neighboring cysteine residues in a folded polypeptide chain often serves to stabilize the three-dimensional structure of the extracellular proteins. Protein hormones such as oxytocin, arginine vasopressin, insulin, growth hormone and calcitonin, all contain disulfide bonds. Enzymes such as ribonuclease, lysozyme, chymotrypsin, trypsin, elastase and papain also have their tertiary structure stabilized by disulfide bonds. Besides the bioactive proteins listed above, there are numerous other proteins that contain disulfide bonds, such as the immunoglobulins (IgA, IgD, IgE, IgM), fibronectin, MHC (major histocompatible complex) molecules and procollagen. Many polypetides from animal venoms also contain disulfide bonds.

In a preferred embodiment, the method of the present invention is used to prepare inactivated forms of neurotoxins, and more preferably neurotoxins from amongst the four groups provided below. As described above, those in Group I typically affect the presynaptic neurojunction, those in Group II typically affect the postsynaptic neurojunction, and those in Group III typically affect ion channels. Lastly, there are also included toxins known only to have a toxic affect by causing membrane damage.

| | |
|---|---|
| Neurotoxins | Membrane-damaging toxins |

| Group I | Group II | Group III | Toxins |
|---|---|---|---|
| notexin | α-conotoxin | dendrotoxins | myotoxins |
| β-bungarotoxin | α-cobrotoxin | scorpion toxins | cardiotoxins |
| crotoxin | erabutoxin | μ-conotoxins | mellitin |
| taipoxin | α-cobratoxin | sea anemone toxins | phospholipases |
| textilotoxin | α-bungarotoxin | | |
| α-latrotoxin | | | |

The method involves a further step of preparing or isolating a corresponding gene (e.g., a cDNA strand) encoding the polypeptide. Using the primary amino acid sequence discussed above, and in view of the present teaching, those skilled in the art will appreciate the manner in which such polypeptides can be synthesized using genetic engineering techniques. Generally, and preferably, one or more of the native control (e.g., leader) sequences of the desired cDNA are removed and replaced with one or more corresponding sequences in order to facilitate the desired expression.

Immunokine components from animal venoms, for instance, can be obtained from the animals themselves or from other sources, or they can be created in the laboratory using conventional protein engineering techniques. In the former approach, animals are induced by mechanical or electrical stimuli to release venom from their glands, which travels through a venom canal and out the fang or stinger. The venom is collected and various constituents of the venom are purified by conventional chromatographic techniques.

In the latter approach, constituents from the venom are synthesized by cloning the genes encoding the various immunokine elements and expressing these genes in heterologous host systems such as bacteria, yeast or higher eucaryotic cell lines. Yeast expression systems are presently preferred, since they tend to provide an optimal combination of such properties as yield and adaptability to human use products.

Expressed products are then purified from any other contaminating host polypeptides by means of chromatographic techniques similar to those used to isolate the polypeptides directly from the venom.

There are significant advantages to the use of host systems other than the venomous animals to obtain the venom components. The danger to human lives in obtaining the venom from the animal is eliminated. There will no longer be a need for the costly animal husbandry required to maintain venomous animals for venom extraction. The quantities of materials that can be obtained from the genetic engineering approach can be one or more orders of magnitude greater than the quantities that can be derived from the venom itself. Moreover, once the gene(s) is cloned and expressed, it can be used to provide a continual, reproducible source in the form of a bacterial, yeast or higher eucaryotic cell line seed culture.

Seed cultures can be stored and transported in the frozen state, lyophilized, or, in some cases, plated on media. Also, the use of genetic engineering tools will enable those skilled in the art to manipulate the genes for the purpose of altering the polypeptide product in any fashion feasible. Using the method of the present invention, in combination with available tools for protein engineering (e.g., site-directed mutagenesis), those skilled will be able to prepare a bioactive polypeptide having any desired level of toxicity, whether non-toxic, or of diminished, equal or greater toxicity than the native form.

The method of the invention provides a further step of expressing the cDNA under conditions in which the polypeptide is recovered in an inactive form due to the failure to form one or more disulfide bridges. As described in greater detail below, this step involves the avoidance of posttranslational processes that would otherwise serve to form such linkages.

Optionally, and preferably, the method provides a further step of treating the immunokines in order to retain the cysteine residues and prevent the spontaneous formation of disulfide bonds. A preferred treatment includes ozone treatment, in the manner described herein. Ozonation affects the cysteine residues by converting the pendent sulfhydryl (—SH) groups to corresponding —SO3X groups, which, unlike the sulfhydryl groups, are unable to form a disulfide bridge. Such treatment is not necessary, however, for those inactivate polypeptides that are found to not spontaneously reform, and that provide the desired activity. Ozonation is preferred for polypeptides such as neurotoxins, where Applicant has shown that upon cleavage and ozonation of the sulfhydryl groups, native neurotoxins are both stable and active.

The invention further provides a bioactive polypeptide that has been rendered inactive by virtue of the failure to form one or more disulfide bridges. Such polypeptides can be stably stored and used under conditions in which disulfide bonds are prevented from spontaneously reforming.

In yet another aspect, the invention provides a method of administering a bioactive polypeptide to a host, comprising the step of providing the polypeptide in an inactive form and within a suitable composition, and administering the composition to a host. In a related aspect, the invention provides a host having administered such a polypeptide. Compositions of the present invention can be used for a variety of purposes. Compositions are particularly useful in situations calling for a polypeptide in a form that is as close to native as possible, yet without an unwanted bioactivity.

Poplypeptides such as the preferred neurotoxins and immunokines can be prepared using genetic engineering techniques within the skill of those in the art, given the present desription. See, for instance, (Fiordalisi et al., (1996) Toxicon 34, 2, 213-224, Krajewski et al (1999) “Recombinant m1-toxin” presented at the 29^{th} Annual Meeting of the Society for Neuroscience) and (Smith et al., (1997) Biochemistry, 36, no. 25, 7690-7996. As the native cobratoxin gene is available, a number of bioengineered variants can be prepared which replace the residues required for disulphide bond formation with other residues. As these amino acid substitutions must be expressed in vivo, the availability of modifications are typically limited to the use of native residues (the standard 20 naturally occurring amino acids) and the host to be employed for expression. In the host, the codon usage will be important in ensuring efficient and maximal expression of the novel protein. Theoretically any amino acid can be substituted for cysteine but as this is a more costly approach to generating immunokine variants relative to synthetic peptide techniques certain residues have been selected which best reproduce the protein characteristics resulting from chemical exposure.

It is preferred to make what are considered to be conservative substitutions, e.g., to limit the cysteine replacement to the following residues; methionine (M), glutamic acid (E), aspartic acid (D), glutamine (Q), asparagine (N), serine (S), glycine (G) and alanine (A). Methionine incorporation can be considered to be the more conservative substitution by replacing one sulphur-containing residue for another. Unlike cysteine, methionine cannot form disulphide bonds. Methionine also reacts readily with ozone to produce the sulfone derivative, therefore the purified product can be exposed to ozone or other chemical agents to confer upon the protein other desirable properties (i.e. low immunogenicity). Also the presence of methionine also allows for the cleavage of the protein into fragments employing cyanogen bromide.

Cleavage of the native cobratoxin and immunokine protein can be achieved with serine proteases (i.e. trypsin) but at sites containing positive residues. This permits also the evaluation and production of smaller peptide fragments for biological activity. The conversion of cysteine to cysteic acid also permits the substitution by other acidic residues such as E, D, Q, N and S. The substitution of E and D for cysteine is estimated to produce a protein with a pI similar to that of alpha-immunokine (pI=4.5). The substitution of cysteine with the residues glycine and alanine would represent standard “neutral” substitutions. A suitable method for creating these genes has been described previously (Smith et al., (1997)). The codon usage of the DNA fragments is optimized for use in commercially used bacterial and yeast expression systems Escherichia coli and Pichia pastoris respectively.

Given the advances in technology in cloning DNA encoding proteins comprising antibodies, the invention also includes DNA which encodes the immunokine of the invention, or a portion of such immunokine. The nucleic acid encoding the immunokine may be cloned and sequenced using technology which is available in the art, and is described, for example, in Wright et al. (1992, Critical Rev. in Immunol. 12(3,4):125-168) and the references cited therein. Further, the immunokine of the invention may be “humanized” using the technology described in Wright et al., (supra) and in the references cited therein.

For example, to generate a phage immunokine library, a cDNA library is first obtained from mRNA which is isolated from cells, e.g., the hybridoma, which express the desired protein to be expressed on the phage surface, e.g., the desired immunokine. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired immunokine, e.g., an immunokine, may be engineered such that the protein is displayed on the surface thereof in such a manner that it is available for binding to its corresponding binding protein, e.g., the antigen against which the immunokine is directed. Thus, when bacteriophage which express a specific immunokine are incubated in the presence of a cell which expresses the corresponding antigen, the bacteriophage will bind to the cell. Bacteriophage which do not express the immunokine will not bind to the cell. Such panning techniques are well known in the art and are described for example, in Wright et al., (supra).

By the term “synthetic immunokine” as used herein, is meant an immunokine which is generated using recombinant DNA technology, such as, for example, an immunokine expressed by a bacteriophage as described herein. The term should also be construed to mean an immunokine which has been generated by the synthesis of a DNA molecule encoding the immunokine and which DNA molecule expresses an immunokine protein, or an amino acid sequence specifying the immunokine, wherein the DNA or amino acid sequence has been obtained using synthetic DNA or amino acid sequence technology which is available and well known in the art.

The invention thus includes a DNA encoding the immunokine of the invention or a portion of the immunokine of the invention. To isolate DNA encoding an immunokine, for example, DNA is extracted from immunokine expressing phage obtained according to the methods of the invention. Such extraction techniques are well known in the art and are described, for example, in Sambrook et al. (supra).

An “isolated DNA”, as used herein, refers to a DNA sequence, segment, or fragment which has been purified from the sequences which flank it in a naturally occurring state, e.g., a DNA fragment which has been removed from the sequences which are normally adjacent to the fragment, e.g., the sequences adjacent to the fragment in a genome in which it naturally occurs. The term also applies to DNA which has been substantially purified from other components which naturally accompany the DNA, e.g., RNA or DNA or proteins which naturally accompany it in the cell.

The invention should also be construed to include DNAs which are substantially homologous to the DNA isolated according to the method of the invention. Preferably, DNA which is substantially homologous is about 50% homologous, more preferably about 70% homologous, even more preferably about 80% homologous and most preferably about 90% homologous to DNA obtained using the method of the invention.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology.

To obtain a substantially pure preparation of a protein comprising, for example, an immunokine, generated using the methods of the invention, the protein may be extracted from the surface of the phage on which it is expressed. The procedures for such extraction are well known to those in the art of protein purification. Alternatively, a substantially pure preparation of a protein comprising, for example, an immunokine, may be obtained by cloning an isolated DNA encoding the immunokine into an expression vector and expressing the protein therefrom. Protein so expressed may be obtained using ordinary protein purification procedures well known in the art.

An inactivated bioactive polypeptide of this invention can also be provided by synthetic means, e.g., solid phase synthesis (also known as combinatorial chemistry). For instance, current technology permits the production of polypeptides such as neurotoxins through peptide synthesis. Many smaller neurotoxins (from conus snails, bee venom and scorpion venom) are routinely produced by synthetic peptide methodology (Hopkins et al., (1995) J. Biol. Chem., 270, no. 38, 22361-22367, Ashcom and Stiles, (1997) Biochem. J. 328, 245-250, Granier et al., (1978) Eur. J. Biochem, 82, 293-299 and Sabatier et al., (1994) Int. J. Pept. Protein Res., 43, 486-495) and some are available from commercial organizations. The above references also describe the synthesis of such peptides incorporating mutant residues (Hopkins et al. (1995) and Sabatier et al (1994)).

Current techniques in peptide chemistry allow for proteins in excess of 80 amino acids can be reliably produced using automated Fmoc solid phase synthesis (ABI 433A Peptide Synthesizer, Perkin Elmer—see www.perkin-elmer.com). Non-native amino acids (acetamidomethyl cysteine, carboxyamidomethyl cysteine, cysteic acid, kynurenine and methionine sulphone) are acquired from Advanced Chemtech (Louisville, Ky.) or Quchem (Belfast, Ireland). Other oxidized or alkylated amino acid variants are available from these agents. The generation of alpha-immunokine is achieved by substituting primarily the cysteine residues (from 1 pair to all 5 disulphide couples) with those residues described above to mimic the effects of ozone and other chemical modifications. Furthermore the substitution of other native and non-native residues for cysteine can be investigated in an attempt to identify immunokine variants with improved biological activity. Also peptide fragments from within the cobratoxin sequence can be created (analogous to Hinmann et al., (1999), Immunoparmacol. Immunotoxicol, 21 (3), 483-506) and examined for receptor binding activity.

Inactivated bioactive polypeptides of this invention can be formulated and delivered in any suitable manner. For instance, for use in treating existing HIV infections, an immunokine will typically be provided in a substantially pure and sterile form, and in a vehicle adapted for delivery. As used herein, the term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state.

To inhibit infection of cells by HIV in vitro, cells are treated with the immunokine of the invention, or a derivative thereof, either prior to or concurrently with the addition of virus. Inhibition of infection of the cells by the immunokine of the invention is assessed by measuring the replication of virus in the cells, by identifying the presence of viral nucleic acids and/or proteins in the cells, for example, by performing PCR, Southern, Northern or Western blotting analyses, reverse transcriptase (RT) assays, or by immunofluorescence or other viral protein detection procedures. The amount of immunokine and virus to be added to the cells will be apparent to one skilled in the art from the teaching provided herein.

To inhibit infection of cells by HIV in vivo, the immunokine of the invention, or a derivative thereof, is administered to a human subject who is either at risk of acquiring HIV infection, or who is already infected with HIV. Prior to administration, the immunokine, or a derivative thereof, is suspended in a pharmaceutically acceptable formulation such as a saline solution or other physiologically acceptable solution which is suitable for the chosen route of administration and which will be readily apparent to those skilled in the art of immunokine preparation and administration. The dose of immunokine to be used may vary dependent upon any number of factors including the age of the individual, the route of administration and the extent of HIV infection in the individual. The immunokine is prepared for administration by being suspended or dissolved in a pharmaceutically acceptable carrier such as saline, salts solution or other formulations apparent to those skilled in such administration.

Typically, the immunokine is administered in a range of 0.1 microgram to 1 g of protein per dose. Approximately 1-10 doses are administered to the individual at intervals ranging from once per day to once every few years. The immunokine may be administered by any number of routes including, but not limited to, subcutaneous, intramuscular, oral, intravenous, intradermal, intranasal or intravaginal routes of administration. The immunokine of the invention may be administered to the patient in a sustained release formulation using a biodegradable biocompatible polymer, or by on-site delivery using micelles, gels and liposomes, or rectally (e.g., by suppository or enema). The appropriate pharmaceutically acceptable carrier will be evident to those skilled in the art and will depend in large part upon the route of administration.

The immunokine (including the corresponding active bioactive polypeptide) of the invention may also be used in a method of screening compounds for anti-HIV activity. A test compound is first screened for the ability to bind to the immunokine of the invention. Compounds which bind to the immunokine are likely to share structural and perhaps biological activities with CXCR4 and thus, may serve as competitive inhibitors for inhibition of the interaction of HIV envelope protein with CD4 and/or CXCR4 plus CD4. An immunokine-binding compound is further tested for antiviral activity by treating cells with the compound either prior to or concurrently with the addition of virus to the cells. Alternatively, the virus and the compound may be mixed together prior to the addition of the mixture to the cells. The ability of the compound to affect virus infection is assessed by measuring virus replication in the cells using any one of the known techniques, such as a RT assay, immunofluorescence assays and other assays known in the art useful for detection of viral proteins or nucleic acids in cells. Generation of newly replicated virus may also be measured using known virus assays such as those which are described herein.

The immunokine of the invention may also be used in competition assays to screen for compounds that bind to CXCR4 and which therefore prevent binding of the immunokine to CXCR4. Such compounds, once identified, may be examined further to determine whether or not they prevent entry of virus into cells. Compounds which prevent entry of virus into cells are useful as anti-viral compounds.

Additional uses for the immunokine of the invention include the identification of cells in the body which are potential targets for infection by an immunodeficiency virus.

By the term “target cell for immunodeficiency virus infection” as used herein, is meant a cell which expresses receptor protein(s) for an immunodeficiency virus and which cell is therefore capable of being infected by an immunodeficiency virus.

Cells which are potential targets for HIV infection may be identified by virtue of the presence of CXCR4 on their surface. The immunokine of the invention facilitates identification of these cells as follows: The immunokine of the invention is first combined with an identifiable marker, such as an immunofluorescent or radioactive marker. Cells which are obtained from a human subject are then reacted with the tagged immunokine. Binding of the immunokine to cells is an indication that such cells are potential targets for HIV infection. The identification of cells which may be infected with HIV is important for the design of therapies for the prevention of HIV infection. For example, CXCR4 is differentially expressed and regulated on human T lymphocytes (Bleul et al., 1997, Proc. Natl. Acad. Sci. USA 94:1925-1930). Further, reactivity of immune cells to MAb 12G5 is high on naive cells and low on memory cells and thus, the pattern of expression of CXCR4 and its utilization by viruses may contribute to immune dysfunction. CXCR4 has also been detected, using the immunokine of the invention, on some endothelial cells (in atherosclerotic plaques), platelets and some hematopoietic precursor cells. In the case of individuals who are infected with HIV, the identification of target cells provides an immune profile of these individuals which provides useful information regarding the progress of their infection.

In addition to the aforementioned uses for the immunokine of the invention, the immunokine is useful for the detection of CXCR4 on a variety of cell types on which CXCR4 may be expressed. For example, CXCR4 is expressed on human neurons (Hesselgesser et al., 1997, Current Biology 7:112-121), including cells in the human brain.

### EXAMPLES

### Example 1

### Isolation of Gland Tissue for RNA Extraction

The following protocol was used to clone the gene encoding α-cobratoxin from the venom of Naja naja siamensis.

(a) Recovery of Venom

Naja naja siamensis snakes were obtained from Siam Farms, Bangkok, Thailand. Animals were shipped to and housed at Ventoxin, Inc., Frederick, Md. USA. The venom glands from N. n. siamensis animals were surgically removed and used to prepare mRNA for generating a cDNA library. Snakes were placed on a schedule for milking (venom extraction). They were milked on day 1 and eight days later milked a second time. On the 2nd or 3rd day, they were anesthetized with sodium pentobarbital and their glands removed (Vandenplas et al., 1985). Gland tissue was quickly cut into small pieces and immediately frozen in liquid nitrogen. Samples were kept at −70° C. until use.

(b) RNA Isolation

Total RNA was isolated from gland tissue by using a standard guanidinium/hot phenol method (Feramisco et al., 1982). Frozen gland tissues (5 g) were placed in a polytron mixer and 10 ml of Solution A (guanidinium isothiocyanate mixture) was added to the tissue. Solution A was prepared by resuspending 100 g of guanidinium isothiocyanate in 100 ml of deionized water, 10.6 ml of 1 M Tris-Cl (pH 7.6), and 10.6 ml of 0.2 M disodium ethylene diamine tetraacetate (EDTA). It was stirred overnight at room temperature.

The solution was then warmed while stirring to 60-70° C. for 10 min to assist dissolution. Any insoluble material remaining was removed by centrifugation at 3000 g for 10 min at 20° C. To the guanidinium isothiocyanate solution, was added 21.2 ml of 20% sodium lauryl sarkosinate and 2.1 ml of β-mercaptoethanol to the supernatant and the volume was brought to 212 ml with water. The final solution was filtered through a disposable Nalgene filter and stored at 4° C. in a tightly sealed, brown glass bottle.

The glands were mixed in the polytron mixer at 4° C. until most of the tissue had been disrupted (about 3-5 min.). The gland solution was placed in a 50 ml polypropylene centrifuge tube and 20 ml more of the guanidinium isothiocyanate mixture was added. The mixture was brought to 60° C. and passed through a syringe fitted with an 18 gauge needle. This shearing technique was repeated 2 to 3 times or until the viscosity of the suspension was reduced. An equal volume of ultra pure liquid phenol preheated to 60° C. was added to the tissue suspension and this was again passed through the syringe 2 to 3 times.

At this point, 0.5 volume of Solution B (0.1 M sodium acetate (pH 5.2), 0.01 M Tris-Cl (pH 7.4), 0.001 M. EDTA) was added to the emulsion and mixed. An equal volume of chloroform/isoamyl alcohol (24/1 v/v) was added and the mixture shaken vigorously for 15 min. while maintaining the temperature at 60° C. The mixture was cooled on ice and centrifuged at 2000 g for 15 min. at 4° C. The aqueous phase, containing the RNA, was recovered and reextracted with phenol/chloroform. To the aqueous phase was added 2 volumes of absolute ethanol and the mixture was stored at −20° C. overnight. All glassware used in extracting and working with RNA had been baked at 250° C. for at least 4 h. Sterile, disposable polypropylene plasticware is essentially free of RNase and can be used for the preparation and storage of RNA without pretreatment.

The RNA was recovered by centrifugation was dissolved in 30 ml of Solution C (0.1 M Tris-Cl, pH 7.4, 0.05 M NaCl, 0.01 M EDTA, 0.2% (v/v) sodium dodecyl sulfate (SDS)). Proteinase K was added to a final concentration of 200 μg/ml and incubated at 37° C. for 2 h. The solution was then heated to 60° C. and 0.5 volume of phenol, preheated to 60° C., was added and mixed vigorously with the RNA-containing solution. Chloroform (0.5 volume) was added to the solution and again mixed vigorously at 60° C. for 10 min. The solution was cooled on ice for 10 min. and then centrifuged at 2000 g for 15 min.

The aqueous phase was recovered and re-extracted one more time with phenol/chloroform at 60° C. The aqueous phase was recovered and reextracted twice with chloroform at room temperature. To the aqueous phase was added 2 volumes of absolute ethanol and put at −20° C. overnight. The nucleic acids were precipitated by centrifugation and the pellet rinsed with 70% cold ethanol. RNA was stored at −70° C. in 70% ethanol until used. When the RNA was ready to be used, it was centrifuged, dried and resuspended in Rnase-free sterile water.

(c) mRNA Purification

Poly(A)+ RNA was enriched by passage over an oligo(dT)-cellulose column using a conventional method (Aviv and Leder, 1972). Commercial oligo(dT)-cellulose was equilibrated with sterile, RNase-free Solution D (0.02 M Tris-Cl, pH 7.6, 0.5 M NaCl. 0.001 M EDTA and 0.1% (v/v) SDS). A 1.0-ml bed-volume of equilibrated matrix was poured into either an Rnase-free disposable polypropylene column or siliconized RNase-free pasteur pipette. The matrix was washed with 3 column volumes of (1) Rnase-free sterile water; (2) 0.1 M NaOH containing 0.005 M EDTA; and (3) sterile water. The column effluent should have a pH less than 8. The column was then washed with 5 volumes of sterile Solution D.

The RNA isolated as described above was heated to 65° C. for 5 min and a 2× concentration of an equal volume of Solution D was added to the RNA solution. The sample was cooled to room temperature and loaded onto the oligo(dT)-cellulose column. The flowthrough from the column was heated to 65° C., cooled to room temperature, and reapplied to the column. The column was washed with 10 volumes of Solution D followed by 4 column-volumes of Solution D containing 0.1 M NaCl. The poly(A)+ RNA was then eluted with 2-3 column volumes of sterile Solution E (0.01 M Tris-Cl, pH 7.5, 0.001M EDTA and 0.05% (v/v SDS).

Typically, NaCl was added to the mRNA to obtain a salt concentration of 0.5 M, and the mRNA was repurified on a second passage over the oligo(dT)-cellulose column using the same procedures as described for the initial column run. Sodium acetate (NaOAc) (3M, pH 5.2) was then added to the mRNA from the second column run to obtain a final concentration of 0.3 M NaOAc. Cold absolute ethanol (2.5 volumes) was added to the mRNA solution and the solution was placed at −20° C. overnight. The N. n. siamensis mRNA was then centrifuged at 12,000 g, the pellet washed with cold 70% ethanol, and stored in 70% ethanol at −70° C. until used. The yield of mRNA from 5 g of gland tissue was 16 μg.

(d) Construction of a N. n. siamensis cDNA Library

Complementary DNA (cDNA) was prepared from 5 μg of N. n siamensis mRNA (Guber and Hoffman, 1983) using commercially-available cDNA synthesis kits. A variety of sources provide cDNA synthesis kits that are useful for such purposes. In this particular case, cDNA synthesis kit, EcoR I/Not I adaptors, T7 sequencing kit, Deaza T7 sequencing mixes, and restriction enzymes were obtained from Pharmacia (Piscataway, N.J.).

A lambda ZAP II/EcoR I CIAP treated vector kit and Gigapack II Gold packaging extract were obtained (Stratagene, LaJolla, Calif.), as was a “GeneAmp PCR reagent kit” (Perkin-Elmer Cetus, Norwalk, Conn.). Oligonucleotides used for screening cDNA libraries and as primers for polymerase chain reactions (PCR) and dideoxynucleotide sequencing were synthesized on a Biosearch 8700 DNA synthesizer by β-cyanoethyl phosphoramidite chemistry and purified on Oligo-Pak columns (MilliGen/Biosearch, Burlington, Mass.).

A protocol for the cDNA synthesis is provided in “You-Prime cDNA Synthesis Kit Instructions”, Pharmacia LKB Biotechnology, the disclosure of which is incorporated herein by reference. (See, in particular, pages 12, 13, 18, 19 and 29 and Procedures A, B and D thereof for the prototypical procedure.) Using procedure B, hemiphosphorylated adaptors containing Not I and EcoR I restriction enzyme sites were ligated to the termini of the synthesized, double-stranded cDNA prepared in Procedure A. After purification of the cDNAs (Procedure D), the N. n. siamensis cDNA were inserted into EcoR I-predigested, phosphatased Lambda ZAP II arms and packaged into viable phage particles by using packaging extracts. The latter was accomplished using a commercially available kit from Stratagene (LaJolla, Calif.) (Catalog #236211, “Predigested Lambda ZAP II/EcoR1 Cloning Kit”).

N. n. siamensis cDNA was ligated to Lambda ZAP II arms using the procedure on page 3 of the Strategene instructions (substituting the test insert for the N.n. siamensis cDNA). The ligated sample was then packaged into viable phage particles using a “Gigapack Gold” packaging extract from Strategene (product insert, page 4). The recombinant bacteriophage was used to infect E. coli host strain, XL1-Blue, which generated the primary cDNA library. The primary library contained approximately 1.35×10⁵ pfu/μg mRNA.

(e) Isolation of α-cobratoxin cDNA from the cDNA Library and Subcloning of cDNA Inserts from Lambda ZAP II Clones

Approximately 100,000 plaques from an amplified cDNA library were analyzed for sequences encoding α-cobratoxin using a degenerate oligonucleotide probe prepared from the known amino acid sequences of α-cobratoxin. The probe (LAS 1) was prepared as follows: 5′- GGI CAI GTI TGT/C TAT/C ACI AAA/G ACI TGG TGT/C GAI GCI TTI TG - 3′

The oligonucleotide probe above was end-labeled on the 5′ end using [³²P]ATP and T4 polynucleotide kinase using standard protocols (Sambrook et al. 1989). The library was screened for the presence of alpha-cobratoxin cDNA on nitrocellulose filters according to standard procedures (Sambrook et al., 1989). Filters were prehybridized for 4 h at 42C in 6× SSC (90 mM sodium citrate containing 0.9 M NaCl, pH 7.0), containing 1× Denhardt's and 100 mg/ml sonicated and denatured salmon sperm DNA. Filters were then hybridized in 4× SSC, pH 7.0, containing 1× Denhardt solution (50×=5 g ficoll, 5 g polyvinylpyrrolidone, 5 g bovine serum albumin/500 ml water) and the radiolabelled oligonucleotide probe for 16 h at 42C.

Successive washes were performed in 2× SSC, pH 7.0, at 37C for 30 min before autoradiography for 16 h at −70C using X-AR film with intensifying screens. Double-stranded cDNA inserted into the multiple cloning site (MCS) of pBluescript SK-contained within lambda ZAP II, were removed as phagemids by an in vivo excision process designed by Stratagene (LaJolla, Calif.) (see Stratagen insert, page 7, “In Vivo Excision Protocol”). Colonies from the in vivo excision were selected by ampicillin resistance, propagated, and the phagemids were isolated by alkaline extraction (see pp. 368-369, “Analysis Lysis Method”). The size of the inserts from the recombinant phagemids were measured on agarose gel electrophoresis after digestion with the restriction enzyme, EcoR I.

(f) Characterization of the Alpha-Cobration cDNA by Asymmetric PCR and DNA Sequencing

The template for asymmetric PCR was double-stranded pBluescript SK-containing cDNA inserts of approximately 400 bp. Oligonucleotides designated as LAS 2 (5′ GAGTTAGCTCACTCATTAGGC 3′) and LAS 3 (5′ ATT-TTCATTCGCCATTCAGGC 3′) were used as primers in asymmetric PCR (see “T7 Sequencing Kit Instructions”, Pharmacia LKB Biotechnology”). Sanger dideoxynucleotide sequencing employed T7 DNA polymerase according to the manufacturer's protocol accompanying the T7 Sequencing (TM) Kit of Pharmacia LKB Biotechnology. N. n siamensis cDNA template, and the primers (LAS 4 and LAS 5) were as described below. Single stranded DNA was used as a template. Programs for sequence analysis from Intelligenetics, Inc. (Mountain View, Calif.), including GENED, SEQ, and IFIND, were used on a VAX from Digital Equipment Corp. (Maynard, Mass.). One of the cDNAs sequences encoded alpha-cobratoxin (identified as Naja naja kaouthia cDNA library clone “NNK III 6.2”). The alpha-cobratoxin cDNA was an incomplete gene in that the leader sequence coding for the snake signal sequence was incomplete (−1 to −20) lacking an in initiation codon (ATG). For purposes of expression, this was immaterial, since the leader sequence was replaced with a functional start codon and restriction enzyme site (as described herein with reference to expression of cDNA in yeast).

The gene encoding alpha-cobratoxin could also have been prepared using the genetic coding sequence for the known amino acid sequence of the protein, and synthetically constructing a suitable gene using automated biochemical techniques.

The PCR-derived DNA was resuspended in TE buffer (20 mM tris-CL, 1 mM EDTA, pH 7.5) and cleaved with the restriction enzyme, EcoR I (see Gibco product insert for EcoR I catalog #15202-013, restriction enzyme assay for EcoR I). The yeast DNA vector (pHILD4) was also taken, resuspended in TE buffer and cleaved with EcoR I.

The vector DNA was cleaved in the same manner as the PCR-derived DNA (see Gibco instructions). After digestion with EcoR I, the PCR-derived DNA and yeast vector DNA was purified by the addition of an equal volume of phenol/chloroform (50/50 v/v), vortexing, and centrifugation in a microfuge (12,000 g). A second chloroform extraction was performed (equal volume of CHCI₃ and sample), vortexing, centrifugation and ethanol precipitation. Ethanol precipitation was performed by adding sodium chloride to the sample (0.2 M final concentration) and 2.5 volumes of cold ethanol. After mixing, the sample was placed on dry ice for 15 min, then centrifuged at 4C in a microfuge (12,000 g) for 15 min. The DNA pellet was dried under vacuum.

Both of the EcoR I-treated DNAs were resuspended in TE buffer and covalently joined together using T4 DNA Ligase (see insert materials, Gibco BRL, Cat. #5224SC, T4 DNA Ligase). The ligated DNA was used to transform competent E. coli cells (see Enclosure 10 for transformation conditions). Transformants growing on TB agar (Terrific Broth+agar) containing ampicillin were isolated and the recombinant DNA analyzed by restriction enzyme analysis.

Optionally, the DNA can be purified from E. coli cells, e.g., in the manner described in “Wizards Maxipreps DNA Purification System”, Promega. Recombinant DNA from clones harboring the α-cobratoxin gene/pHILD4 construct was used for integration into the yeast, Pichia pastoris.

(g) Cloning and Cytoplasmic Expression

Expression of the alpha-cobratoxin gene in the vector, pHILD4 yields a cytoplasmic product that lacks posttranslational modifications, including disulfide bond formation.

Suitable techniques for cloning and expressing genes into Pichia pastoris have been developed by the Phillips Petroleum Company and compiled in “Pichia Expression Kit—A Manual of Methods for Expression of Recombinant Proteins in Pichia pastoris”, which was prepared by Invitrogen and accompanies their expression kit having catalog #K1710-01.

The gene encoding alpha-cobratoxin from amino acids +1 to +71 can be removed from the cDNA by using the following polymerase chain reaction primers:

(a) 5′ sense primer (LAS 4)=5′-GGATCC GAATTC ACG atg [ATA AGA ACA]-3′ (36 mer) and

(b) 3′ antisense primer (LAS 5)=5′-CCTAGG GAATTC TTA TCA [AGG a TGG]-3′ (36-mer).

Recombinant DNA prepared as described herein was treated with Sst I restriction enzyme under the same reaction conditions as described above with respect to EcoR I, except using reaction buffer No. 2 described in the above-captioned Gibco EcoR I product insert. The restricted DNA is purified by the addition of an equal volume of phenol/chloroform (50/50 v/v), vortexing, and centrifugation in a microfuge (12,000 g).

A second chloroform extraction was performed (equal volume of CHCI₃ and sample), vortexing, centrifugation and ethanol precipitation. Ethanol precipitation was performed by adding sodium chloride to the sample (0.2 M final concentration) and 2.5 volumes of cold ethanol. After mixing, the sample was placed on dry ice for 15 min, then centrifuged at 4° C. in a microfuge (12,000 g) for 15 min. The DNA pellet was dried under vacuum and resuspended in TE buffer.

The DNA pellet is then integrated into the chromosome of Pichia pastoris strain GS115 using conventional procedures for integrating genes into Pichia pastoris (e.g., p. 29-38, “Growth of Pichia for Spheroplasting”) and expressing the integrated genes (pp. 41-45, “Expression of Recombinant Pichia strains”).

### Example 2

### Recovery and Yield

A fermentation of a cytoplasmically-expressing clone harboring the gene encoding α-cobratoxin can be performed in a 5 L New Brunswick BioFlo III fermentor. The size of the fermentation can be scaled up or down depending on the requirement for product. For a 5 L batch, a frozen seed culture containing the alpha-cobratoxin construct is used to inoculate 10 ml of MGY media (see attached media recipe) in a test tube. After 18 to 20 hours growth at 30° C., 0.5 ml is used to inoculate 50 ml of MGY in a 250 ml flask. After 36 to 38 hours of growth, the entire 50 ml is used to inoculate the 5 L fermentor. The fermentation is performed in a basal salt medium with 26.7 ml 85% phosphoric acid, 0.93 g/L calcium sulfate-2H₂O, 18.2 g/L potassium sulfate, 14.9 g/L magnesium sulfate, 4.13 g/L potassium hydroxide, 40 g/L glycerol and 2 m/L of basal salts (PTM) are added. PTM basal salts consist of 2.0 g cupric sulfate, 0.08 g sodium iodide, 3.0 g magnesium sulfate, 0.2 g sodium molybdate, 0.02 g boric acid, 0.5 g cobalt chloride, 7.0 g zinc chloride, 22 g ferrous sulfate, 0.2 g biotin and 1 ml sulfuric acid per liter. The fermentation culture is fed with a 50% solution of glycerol in deionized water, while the methanol feed solution is 100% methanol with 2 ml of PTM basal salts and 1 mg biotin per liter. “Structol” brand antifoamer can be used as antifoam control; the pH during the glycerol phase is maintained at pH 5.0 using 30% ammonium hydroxide; dissolved oxygen is controlled above 25% saturation by supplementing with pure oxygen.

A standard fermentation procedure is followed which includes an initial batch phase followed by a 4 hour glycerol fed-batch with a feed rate of 15 ml/L/h of a 50% glycerol solution. At the completion of the glycerol fed-batch phase the methanol induction phase is started. The rate of methanol feeding is increased gradually from 3.5 to 12 ml/L/h within 6 to 8 hours and maintained at 12 ml/L/h. Samples are taken during fermentation for measuring optical density at 600ₙₘ, cell dry weight and SDS-PAGE analysis.

Yeast cells are recovered from the fermentation by centrifugation. Cells are washed in breaking buffer (50 mM NaH₂PO₄, 1 mM EDTA, 5% glycerol, 1% PMSF, pH 6.0), and resuspended in the same buffer prior to disruption in an APV Matnon Gaulin 30CD pilot scale homogenizer. Cell debris is removed by centrifugation and a PEI precipitation is performed on the cell extract in order to remove endogenous nucleic acids,. Polyethyleneimine (PEI) (10%) is added to the cell extract to obtain a final concentration of 0.4% PEI. The mixture is allowed to sit for 3 to 5 hours at 4C with stirring. The mixture is centrifuged at 27,000×g for 15 min and the supernatant is dialyzed against 50 mM NaH₂PO₄, pH 6.0 at 4C. The recombinant product is purified by ion exchange (e.g., cationic exchange matrix) and molecular sieve chromatography.

There have been a number of heterologous proteins produced using the Pichia pastoris expression system. The levels of expression from intracellularly expressed proteins has ranged from 0.3 to 12 g/L depending on the protein expressed (Biotechnology 11, 905-910 (1993)). The level of expression is usually dependent on such factors as the genetic construct itself, cell copy number and fermentation optimization (e.g., cell density, optimal pH and dissolved oxygen concentration). Yields from an alpha-cobratoxin gene expressed intracellularly in Pichia pastoris will typically fall in the range stated above.

### Example 3

### Ozonation

Ozone (O₃), a powerful oxidant, is used for water disinfection. In the course of the present invention, ozone treatment is preferably used to treat the recovered, inactive polypeptide in order to render it incapable of spontaneous reformation. Optionally, ozonated pure water can be used to itself selectively break the disulfide bonds of a formed polypeptide in order to provide an inactive, denatured, and stable form thereof.

Ozone treatment can be used to quickly provide microbial sterilization and disinfection, organic compound destruction, and conversion of iron or manganese salts to insoluble oxides which can be precipitated from the water. The major reaction byproducts are water, oxygen and carbon dioxide. For environmental and safety concerns, unreacted or residual ozone should be monitored. A number of UV spectrophotometric methods can be used to determine the level of ozone in water or physiological saline. Ozone has an absorption peak at 260 nm whereas oxygen does not absorb at this wavelength. When ozone concentration was measured ice water (1° C.±1° C.) by three different colorimetric methods, the absorbance coefficient in ozone at 260 nm as A_{1 cm}^{1 mg/L} is 0.11.

A wavelength scan of ozonated water was determined at various dilutions. Using the same ozonated water, the ozone concentration was determined by Accuvac method described below. Using this, or similar methods, it is possible to calculate the ozone content of the ozonated water in mg of O₃/L.

A standard curve for the ozonated water was also prepared. From this curve one can derive the absorbance coefficient of ozone in any given solution. Table 1 below provides a representative relationship between absorbance coefficients and concentration for ozonated water.

Absorbance coefficient (A)^{mg/l}=(Absorbance at 260 nm)÷(Concentration of Ozone)

TABLE 1

| Absorbance | | Absorbance |
|---|---|---|
| of Ozonated | Concentration of Ozone by | Coefficient of |
| water at 260 nm | Accuvac method mg/L | Ozone at 260 nm |
| 1.5717 | 13.48 | 0.11659 |
| 0.628 | 6.44 | 0.0975 |
| .39822 | 2.908 | 0.1369 |
| .25953 | 2.6792 | 0.0968 |
| .19797 | 1.722 | 0.11496 |
| .13605 | 1.28 | 0.1062 |
| | AVERAGE VALUE | 0.11 |

Three different calorimetric methods (“Accuvac”, “Alizarin” and “Indigo Trisulphonate” methods) were used for the determination of ozone concentration in ice water (1° C.±1° C.), and compared to absorbance at 260 nm. Ozonated water was prepared as described in above. Certain of these methods are used by the International Ozone Association Standardization Committee.

### METHOD 1

### Alizarin Method

The method is directly applicable in the range of 0.03 to 0.6 mg/L. A stock solution of Alizarin violet 3R is made up as a 0.2 mM solution. Disperse 124.45 mg of the dye into an aliquot of distilled water in a 1 liter volumetric flask. Mix magnetically overnight. Add 20 mg of analytical grade sodium hexametaphosphate, 48.5 g of analytical grade ammonium chloride and 1.6 g of ammonia expressed as NH₃. Dilute with distilled water to 1 liter and stir overnight. A 10-fold dilution of this solution has an absorbance of 0.155 cm⁻¹.) 20 ml of the reagent solution is introduced into each of two 200 ml volumetric flasks. Fill one flask with ozone free water. Fill the other flask with the sample water by introducing the sample below the surface of the dye solution to prevent ozone loss by degassing. When measured, the difference in absorbance at 548 nM is 2810 L/M/cm. This equates to the expression:

mg/L O₃=Total volume (200 ml)×(change in absorption)÷(Cell length (1 cm)×0.059×volume of sampled water (180 ml))

### METHOD 2

### Indigo Trisulphonate Method

The method is directly applicable in the range of 0.01 to 0.1 mg/L of ozone in water. A stock solution of indigo-trisulphonate is made up as a 1 mM solution by dispersing the dye into a solution of analytical grade phosphoric acid at a concentration of 1×10⁻³ M. A 100-fold dilution of this solution has an absorbance of 0.16+/−0.01/cm at 600 nm and should be discarded if the absorbance is lower than 80% of the starting value. Normal stability lasts one month. As a diluted reagent, 20 ml of the stock solution is diluted to 1 liter together with 10 g of analytical grade NaH₂PO₄ and 7 ml concentrated analytical grade H₃PO₄ (stability of the diluted solution: one week).

In use, 10 ml of diluted reagent solution is introduced into each of two 100 ml volumetric flasks. Fill one flask with ozone free water (e.g. distilled water). Fill the other flask with the sample water by introducing the sample below the surface of the dye solution to prevent ozone loss by degassing. Measure the difference in absorbance at 600 nm between blank and sample with 5 or 10 cm cells. The measurement is to be made as soon as possible but preferably within 4 hours. The pH value of the measured solution must be lower than 4.

The proportionality constant is 0.42+/−0.01/cm/mg/L ozone, which is equal to a difference in absorbance of 20 L/M/cm (Stoichiometry is considered as 1:1).

mg/L (O₃)=(total volume (100 Ml)×Change in absorption)÷(cell length (cm)×0.42×Volume of sampled water (90 ml))

### METHOD 3

### Accuvac Method

As ozone reacts quantitatively with indigo trisulfonate (Blue indigo dye), the color of the solution fades. Color intensity is inversely proportional to the amount of ozone present, is then measured at 600 nm with a spectrophotometer. The reagent is formulated to prevent interference from any chlorine residual which may be present. The method is directly applicable in the range of 0 to 0.25 mg/L.

In use, gently collect at least 40 ml of sample in a 50 ml beaker. Collect at least 40 ml of ozone free water (Blank) in another beaker. Fill one Indigo ozone reagent Accuvac ampule with the sample and one ampule with the blank. This is done by immersing the ampule in the beaker which has the sample. Quickly invert the ampules several times to mix. Take an aliquot of the samples and read at 600 nm in spectrophotometer.

Read a blank value as X at 600 nm. 0.125 mg/L O₃ should have absorbance of x/2 g/L of O₃=(0.125×O.D. of the blank value/2)÷(O.D. of the sample at 600 nm×Dilution factor).

TABLE 2

| | OZONE CONCENTRATION | |
|---|---|---|
| METHOD | (mg/L of water) | NOTE |
| Accuvac | 13.676 | |
| Alizarin | 16.8 | |
| Indigo - | 15.85 | |
| Trisulphonate | | |
| UV absorption | 15.45 | (Abs/A^{1 cm/mg/l}) 1.710/.11 |
| at 260 nm | | |

Table 2 shows the ozone concentration, as determined by these various methods, for aliquots of the same ozonated water. From the results in TABLE 2 it can be seen that each method provides substantially the same concentration of ozone. Since all the four methods seem to be comparable to each other, the UV absorption method is preferred since it is simple and inexpensive to perform.

Ozone was produced by a high voltage discharge using Tri Atomic Oxygen Generator (Model No. 3, Serial No. 34 from modern Medical Technology Boca Raton, Fla.) The oxygen was passed through the generator to produce the ozone. Approximately 0.2% of ozone was produced in the equipment at the rate of bubbling used (about 200 ml/min). However, for quantitation studies a sample was taken with each series of experiments.

Absorption measurements were made in the Beckman DU 650 Spectrometer using cm quartz cuvettes. A standard curve was obtained by serially diluting the ozonated water and measuring the absorbance at 260 nm. The standard curve was also obtained by using a calorimetric method using commercially available Accuvac ampules (From Hach, P.O. Box 389, Loveland, Colo. 80539)

Saturated ozone water was prepared in the following manner. Oxygen was bubbled at the rate of 200 ml/min to ice water (1° C.±1° C.). The container with distilled water was kept in an ice bath during the ozonation. Ozone, bubbled into water or saline, was determined by measuring the absorbance at 260 nm. Using a 50 mL sample, it takes a minimum of 30 minutes to reach an absorbance reading of 2.0, although the time is dependent upon the oxygen input.

Since water that is saturated with oxygen will not become saturated with ozone, the flow rate of input oxygen was ideally kept at equal to or less than 200 mL/min. Once the ozonated water reaches an absorbance of 1.0 to 2.0, serial dilutions of the ice cold ozonated water were made and measurements of the absorbance at 260 nm were made. The ozonated water was also used to measure kinetics, and in particular, decay rate over the time. The serially diluted water was used to measure the ozone concentration by Accuvac method.

Water ozonated in this manner can be used to oxidize a formed polypeptide, in order to cleave the disulfide groups and render the polypeptide inactive. Alternatively, and preferably, the ozonate water can be used to stabilize a polypeptide that is prepared in an inactive form by the genetic engineering method described above. In either case, the oxidized peptide can be compared to the original, active toxin using a variety of methodologies, including animal models and bioassays.

In a typical approach, the material to be stabilized (e.g., lyophilized salt free toxin) is weighed into 150 ml plastic bottles, each containing 600 mg of toxin. Approximately 800 ml of pure deionized water is allowed to chill in the freezer until ice crystals begin to form. The beaker of pure water is placed in an ice bath and ozonated by bubbling O₃ from an ozone generator connected to an O₂ source. Measurements of OD are taken at 260 nm using a 1 cm light path until an OD₂₆₀ of 2.0 is achieved.

Sixty ml of ice cold ozonated pure water is added to each bottle containing 600 mg of toxin, resulting in a 1 percent solution (a concentration of 10 mg/ml). While waiting for the powder to dissolve, the bottles are stored in the freezer and ice crystals are again allowed to form. Once in solution, the bottles are placed in an ice bath where each bottle is ozonated for 30 seconds by bubbling ozone into the solution. Ten bottles are done at one time, such that each bottle is ozonated for 30 seconds every five minutes. This is done to maintain an effective level of O₃ and is continued for seven hours.

Periodic testing is done by injecting mice with the toxin suspension and monitoring the time to death. When the mice no longer die (after seven hours ozonation) all disulfide bonds have been broken, and the material has been effectively converted from an active toxin to an atoxic toxoid.

It has been noted that if the original ozonated protein solution is maintained at 4° C. for 24 hours and, no further ozonation is carried out, the disulfide bonds are likely not going to be broken, and the solution will remain toxic and able to kill mice. Also, when bacterial or viruses suspensions are added to ozonated water as prepared above, there is immediate 6-8 log kill. Since bacterial and viral kill appears to occur well before oxidation of proteins, ozonated water prepared in this manner can be used to treat protein-containing formulations (e.g., monoclonal antibody preparations) in order to inactive any remaining animal viruses without damaging the antibody itself by breaking critical disulfide bonds.

The oxidized (or stabilized) toxin polypeptide can be compared to the native alpha neurotoxin in a number of respects. It is found that the former is atoxic is mice, while the latter retains full toxicity. The molecular weights as measured on SDS gels are 7380 daltons for both the primary neurotoxin and the resultant oxidized peptide. The isoelectric point as measured by iso-electric focusing gels varies substantially because of the breaking (or stabilized failure to form) of the five disulfide bonds creating a net charge change of ten. The isoelectric point is the pH at which a protein migrates to in an ampholyte solution (continuous pH gradient) to which a current is applied. The primary alpha neurotoxin and resultant oxidized peptide also show separate peaks when measured by HPLC and FPLC.

### Example 4

### Immunokine Production

A preferred process for the production of an immunokine of this invention is outlined below. Alpha-immunokine-NNS (immunokine) is a protein derived from alpha-cobratoxin. Cobratoxin (CTX) has a molecular weight of 7821 and is composed of 71 amino acids. The native protein is purified from the venom of the Thailand cobra, Naja naja siamensis. Alpha-cobratoxin from the Thailand cobra (Naja naja siamensis) was purchased from Biotoxins, Kississimi, Fla. The published amino-acid sequence for cobratoxin employing single letter code is:

ICRFITPDITSKDCPNGHVCYTKTWCDAFCSIRGKRVDLGCAATCPTVKT
GVDIQCCSTDNCNPFPTRKRP

Employing the reactive molecule, ozone, the precursor protein is modified through the addition of oxygen molecules. Ozone has the major advantage in that when the reaction is complete there is no residual material which requires removal. Unreacted ozone decays back to oxygen in a relatively short period of time.

The procedure below describes the dissolution of ozone into saline (0.9%) and its addition to cobratoxin to form immunokine. The reaction is rapid being completed in minutes. In order to create a more homogeneous product consistently the procedure described below was developed whereby an ozone-saturated fluid is added directly to a solution of cobratoxin. It is expected that greater reproducibility can be achieved with this method. The critical point of the reaction centers on adding sufficient ozone to ensure that no native cobratoxin remains. When the reaction is deemed complete several parameters can be measured to be suggestive of successful preparation. The reaction can be conducted at ambient temperatures but the concentration of the final product is limited to below 350 mg/ml. This arises because of the limitations placed on dissolving ozone in saline at these temperatures.

### MATERIALS

Equipment

Approved ozone generator—Haemozone or equivalent

Spectrophotometer—Beckman or equivalent

Peristaltic pump, digital input

Thermometer, degrees centigrade, range minimum of −5° C. to 25° C.

Pipette, 1 ml Gilsen or equivalent or disposable (5 ml)

Quartz cuvette or similar, non absorbing at 260 nm

Glassware, depyrogenated and autoclaved, flask or graduated cylinder appropriate for reaction volume, minimum of 2 required.

Insulated container capable of holding chosen glassware (optional)

Consumables

Gloves, disposables

Oxygen, medical (USP)

Saline, 0.9% for injection from approved source

Cobratoxin, from approved source

Disposable filters, 0.2 μm for bacterial culture

Icepacks chilled to −20° C. (optional)

Ice (optional)

Confirm that ozone generator has recently been validated for function and output. Turn on oxygen supply at outlet valve. Switch on ozone generator. Adjust oxygen flow at regulator to give a flow reading on generator of up to 0.25L per minute. Switch on or ensure sparking coil is operational (listen for auditory beep). Switch on peristaltic pump and set flow to 10-15 ml per minute. Inspect tubing for defects. Attach bubbling frit to peristaltic output and place in container of clean water. Ensure frit output is functioning and satisfactory. Confirm that ozone production has commenced and is rising. Allow machine to operate for 30 minutes in order to warm-up.

Switch on spectrophotometer and/or UV lamp and allow to warm-up for 20 minutes. Set absorbance measurements at 260 nm. The machine should be blanked against an aliquot of saline (see below). With gloved hands clean frit surface with alcohol and place in saline. Increase peristaltic pump flow to 15 ml per min. The object is to supply as much ozone to the solution without inhibiting ozone production. At 10 minute intervals remove aliquots from the solution with a gilsen pipettor or disposable pipette and record the absorbance at 260 nm.

1. Chill saline to below 3.5° C. prior to commencing. This may be achieved by storing saline solution at a suitable temperature. If saline temperature is not sufficiently low the solution can be stored in a −20° C. refrigerator until the saline has reached a temperature of −5° C. or below. Do not freeze the saline solution solid though the presence of slush is quite acceptable.

2. While wearing gloves weigh-out cobratoxin either as 600 mg lots or prepare a 60 mg/ml solution in saline for injection.

3. Add 10 ml solution to depyrogenated 1L (or greater) flask. For larger volumes add 10 ml of 60 mg/ml cobratoxin per liter. Appropriately sized containers should be employed.

4. Commence addition of ozone to saline which is below or being held at a temperature of 3.5° C. or below. Monitor ozone content in saline until the absorbance at 260 nm is recorded at above 1.95 and below 2.05. Place alcohol-cleaned thermometer in saline, measure temperature and record. If the saline temperature exceeds 4.0° C. abandon process and return saline to refrigerator for further chilling. Should the 260 nm reading reach above 2.1 then allow the saline solution stand at room temperature until it has decreased to within the correct limits.

5. Immediately add ozone treated saline up to the correct mark on the flask containing the cobratoxin solution. Alternatively remove sufficient saline from the ozone solution to leave 990 ml. Mix by agitation and store overnight on the bench (>18 hours). If volumes greater than 1L are being prepared, ozone-treat the quantity desired and add to greater volume flasks. Do not make sequential 1L lots from the same ozone treated solution unless it is confirmed by spectrophotometric means that the 260 nm limits before each addition are satisfied.

6. Following overnight storage record pH of solution, perform spectral scans from 215 nm to 305 nm and calculate the 260/280 ratio. Toxicity can be determined by injecting 1 ml (600 ug) into at least 2 mice via the intra-peritoneal route. For this purpose a 27 gauge, 0.5 inch insulin syringe is preferred. The mice should be monitored for 24 hours. Alternatively or concurrently the absence of cobratoxin can be demonstrated by chromatographic analysis.

7. Remove 10 ml aliquot for retention and place in sterile glass vial, seal, crimp and label.

8. Benzalkonium chloride can be added to a final concentration of 0.01%.

9. (Remove aliquot (1 ml) with pipette or syringe and place in sterile container for analysis by mass spectrometry.)

Spectrophotometric scans of the ozone treated cobratoxin from 200 nm to 400 nm were identical to those described by Chang et al. (1990) confirming the modification of the tryptophan residue. Because ozone attacks tryptophan there is a significant reduction in the UV absorption at 280 nm—approximately half that measured for the original cobratoxin solution and an increase in the absorption at 260 nm. This provides a simple method to determine if the chemical modification is sufficiently complete to produce a satisfactory product. If the A260 value is divided by A280 a ratio is developed. From our experience and validation, if the ratio is greater than 2.7 and the pH is 4.5 or less then the product is non-toxic. This approach towards an indication of potency is appropriate only for those proteins which have tryptophan residues. However it should be noted that there exists a fluctuating curve for the ratios which peak at 3.4 before dropping to levels below 2.6 and rising again. At this point the product is being deteriorated and fragmented by excess ozone. It is therefore best to combine these measurements with other assays for potency and/or toxicity. Potency of the modified neurotoxin was evaluated through a modification of the procedure described by Stiles et al. (1991).

The reaction can be conducted at room temperature if refrigeration is unavailable but the concentration of final product will be substantially less (approximately 300 mcg/ml). This results because the solubility of ozone in saline is dependent on the temperature of the liquid. The lower the temperature the higher the ozone concentration and subsequently the greater the quantity of material that can be reacted at one time. To all intents and purposes the product produced at 300 mcg/ml and 600 mcg/ml with the appropriate levels of ozone were identical and it is known that material produced at ambient and chilled temperatures by the previous bubbling method do not differ by mass spectrometry and sequence. The reaction is a single step one, easily reproducible and provided the correct conditions were employed it can be reasonably assumed that the drug produced is at the desired potency.

An immunokine solution prepared in this manner had an acidic pH and a pI of approximately 4.5. Cobratoxin solutions are basic having pH of 8.5. In solution, the drug migrates through molecular sieving gels as monomers, dimers and tetramers. Cobratoxin migrates under these conditions as a monomer. Upon analysis on NuPAGE (Novex) SDS polyacrylamide gel electrophoresis (PAGE) the cobratoxin migrates as a 14 Kd and 8 Kd protein with a reference to comparable proteins under unreduced and reduced conditions respectively. Immunokine migrates under reduced and unreduced conditions without change. A single protein band is not obtained showing a diffuse smear from the loading gel down to a molecular weight equivalent to 8 Kd. Additionally, the protein is resistant to staining with standard coomassie dyes. By ion exchange, cobratoxin and immunokine have generally opposite properties consistent with the proteins' charges. Specialized ion-exchange chromatographic resins and conditions can be employed to confirm the retention of positive charges which are considered critical for neuroactive properties.

As defined by mass spectrometry the average molecular weight of immunokine is 7,933.3±30 daltons (determined from 7 lots, 5 consecutive assays each) with a molecular weight range of 7,600 to 8,400 daltons. This molecular weight variance is expected by the nature of the reaction and ozone. As indicated above excessive ozone application can fragment the protein and insufficient levels do not modify enough amino-acid residues to render the neurotoxin atoxic. The calculated average molecular suggests the addition of 6 oxygen residues with higher molecular weights having correspondingly more. Smaller than expected molecular weights suggest protein fragmentation. Current analytical techniques allow for limited structural identification of the number and location of oxygen residues being added to the protein and rely heavily on previously published information and current chemical theory. Amino acid analyzers do not recognize unnatural amino acids and have limited capabilities for this application.

### Example 5

### FeLV Study

A group of 87 that had tested positive for either FeLV or FIV yielded 87 was studied. Of these 87, 20 were found to be negative for both FeLV and FIV when blood samples were submitted to the University of Miami Medical School Laboratory. These 20 were excluded. Fourteen cats presented in critical condition were also excluded. All of these cats died within ten days. The study therefore became a study of cats with chronic FeLV and/or chronic FIV. Confirmation of presence of either or both viral infections in each cat was determined by tests conducted by the University of Miami Medical School Pathology Reference Laboratory by either IFA or ELISA tests.

Thirty-seven cats were confirmed positive for either FeLV, FIV, or both as follows: twenty-eight only FeLV, seventeen only FIV, seven both FeLV and FIV. Hematocrits ranged from twenty-eight to forty with a median range of thirty to thirty-five and there were no consistent abnormalities in the white cell counts or differentials. The occasional cat would have a slightly elevated segmented neutrophil count and/or aslightly decreased lymphocyte count. Physiological abnormalities include poor appetite resulting in weight loss, poor hair coat, diminished activity, frequent and sometimes continuing bouts of rhinitis and/or sinusitis, gingivitis, frequent abscesses. Interestingly, two cats were in excellent health with glossy hair coats, normal to slightly above normal weight, normal strength and activity, etc. Both of these cats had been vaccinated for FeLV as young adults and were only mildly positive to tests for FeLV. Cat owners were instructed as to how to give the mCTX injections and a quantity sufficient for thirty days was dispensed.

Each cat was given a physical examination and the results recorded. History included length of known infection and/or when cat was first discovered to be FeLV or FIV positive, previous or current therapy. Blood was drawn for CBC and test for FeLV and FIV. Criteria for entering the study was either IFA or ELISA positive as determined by Pathology reference Laboratory. Ploymerase Chain Reaction (PCR) testing was carried out by Dr. James Thompson, D. V. M. (University of Florida Veterinary Teaching Hospital). At the end of each thirty day period blood samples were submitted for CBC's including differentials, and FeLV and/or FIV tests. Placebo controls were not utilized since these animals were privately owned. The animals were monitored for FELV and FIV by ELISA and these acted as their own control in the objective sense due to absence of the “placebo” effect., subjectively, improvement was noted in found

The following concentrations were used to determine the IC50 FIV values for MCTX; no MCTX (control), 0.1, 0.4, 1, 4, 10, 20, 50, 100 and 200 ug/ml. Results are given in FIG. 1. Using the linear portion of the graph in FIG. 1 and IC₅₀ value of 804 ug/ml was determined. It should be noted that the concentrations used in the determination were well below the calculated IC₅₀ concentration, however, due to the scarcity of material another IC₅₀ determination was not possible. These data suggest that modified cobratoxin may not block effectively over 4 days.

The data presented in FIG. 2 shows the infectious virus yield over a four week period. These data show that the total virus formation from cultures treated with MCTX were reduced compared to cultures with no drug. FIG. 3 is a re-plot of data from FIG. 2, showing tha percent inhibition of virus from cultures treated with MCTX compared to no drug control. From these data both concentrations of MCTX appear somewhat effective over 4 weeks.

The approach to treating infected cats was empirical. To avoid any possible adverse reactions to the MCTX, it was decided to administer small doses initially though the in-vitro testing indicated that higher doses would be required. Also, positive responses were seen in various animals with low concentrations of MCTX (Harrison, 1989 and Smith, 1991). As the MCTX appears to have broad anti-viral properties, cats presenting with FeLV were included to evaluate if the MCTX could be utilized against other lentivirus infections. The treatment regime began with 5 micrograms of MCTX every 12 hours by subcutaneous injection for a period of thirty days. At the end of the thirty days, tests for FeLV/FIV were to be conducted and compared with pre-treatment tests. Following thirty days of twice daily treatment the first group of cats returned for clinical appraisal and blood samples. In every case there were clinical improvements such as increased appetite, weight gain, improved hair coat, more playful, etc. There were no significant changes in IFA and ELISA titers after thirty days of treatment. Repeat blood tests were scheduled thirty days later. At the end of the second thirty day period (30 days from last treatment) all in-house tests were still positive and were confirmed positive at the University of Miami Medical School Laboratory. Clinical improvements, however, were being maintained without further treatment.

At this phase it was decided to resume treatments and to increase the dosage to 10 micrograms every 12 hours and continue as long as necessary to obtain negatives or until the cat owner elected to drop out. The laboratory reported the IFA titers for FIV as 1:50 (borderline negative), 1:250, 1:500. IFA or ELISA for FeLV was subjectively reported as 1, 2, or 3 plus depending on depth and rapidity of color change in the tests. At this dosage level we began to see some reduction in titers after each thirty days of treatment. Meanwhile all cats in the “chronic” study continued to do well and each month one or more owners elected to drop out either because of satisfaction with clinical results or the inability to continue twice daily injections to the cat.

The next dosage increase was to 25 micrograms every 12 hours. After one month at this level the first negative for FeLV was attained, both for IFA and ELISA, in a cat that had been positive for both FIV and FeLV. The cat remained positive for FIV. Unfortunately, this owner dropped out after the FeLV negative tests. A second cat positive for both FIV and FeLV tested negative for both viruses at the end of the second month of the 25 microgram dosage. At this point a decision was made to double the dose each month until more negatives were attained. To date all cats that have remained in the study, both FIV and FeLV, have become completely negative with the exception of five cats that are IFA negative and ELISA positive for FeLV. All five of these cats have gone through at least 30 days of 200 micrograms every 12 hours. Three of them finished 60 days at this level. Only one owner reported a troublesome side effect. This cat was FIV positive. After three or four days of treatment the owner reported the cat had developed diarrhea. Treatment was discontinued for a few days and the diarrhea subsided. Treatment was resumed and the diarrhea started again. Lactobacillus was prescribed twice daily. The diarrhea stopped and treatment was continued uneventfully.

From Table 1 the results can be summarized. From 28 chronic FeLV cats, fourteen stopped treatment by owners due to satisfactory clinical improvement in their condition. Fourteen went to IFA negative. Nine of these also went to ELISA negative while five remained ELISA positive. The laboratory reported these as weak positives. Of interest here also, the two cats that had been vaccinated against FeLV as young adults remained ELISA positive. Five cats tested PCR negative for FeLV. From 24 cats with FIV, seventeen with FIV alone plus seven with both FIV and FeLV, fourteen dropped out after satisfactory clinical improvement. Ten of the remaining ten went to IFA negative. ELISA testing was not done in the last months of the study on the FIV cats.

TABLE 1
Summary of Dosage Regime and Blood Analysis
from 52 Cats with FeLeuk and/or FIV

| | | | | Cumulative FeL/FIV | | |
|---|---|---|---|---|---|---|
| | Dosage | Total drug | Losses | (negative) | | |
| | (μg/ml) | Administered | from | ELISA | | FeL |
| Time (months) | B.I.D) | (ug/cat) | study | IFA | | (−ve) PCR |
| 1 | 5 | 300 | — | 1 | 1^{a} | nd |
| 2 | 5 | 600 | — | 5 | 1 | nd |
| 3 | 10 | 1200 | — | nd | 2 | nd |
| 4 | 25 | 2700 | 3 | 7 | 3 | nd |
| 5 | 25 | 4200 | 28 | nd | nd | nd |
| 6 | 50 | 7000 | 28 | 7^{b} | 6 | 1 |
| 7 | 100 | 13000 | 28 | 9 | 8 | 2 |
| 8 | 100 | 19000 | 28 | 19 | 24 | nd |
| 9 | 100 | 25000 | 28 | 19 | 24 | 5^{c} |
| 12 | 100 | 31000 | 49^{d} | 19 | 24^{e} | nd |
| Totals | — | 31000 | 49 | 79% | 100% | 20% |

nd = not determined,
^{a}Cat vaccinated with FeLeuk,
^{b}Previously negative cat tests positive,
^{c}Random data, not performed on all cats,
^{d}3 cats remained in long term study (18 months) to observe ELISA responses,
^{e}Includes 3 cats positive for FeLeuk and FIV.

Dosage values (given IM) are per animal irrespective of size. Percent values calculated from animals remaining at end of trial(24). Three of the above cats had concurrent chronic conditions. Two of these cats had FeLeuk titers to both ELISA and IFA testing although they had been previously vaccinated for it.

TABLE 2
Summary of Response in 52 Cats with Feline Leukemia and FIV

| | | | | | |
|---|---|---|---|---|---|
| Duration of | | | | | |
| Therapy | Improved | Weight | Increased | Consumer | Consumer |
| (months) | appetite | Gain | Activity | Satisfaction | cessation |

| | | | | | |
|---|---|---|---|---|---|
| 1 | 52 | 43 | 52 | 52 | — |
| 2 | 52 | 45 | 52 | 52 | — |
| 3 | 52 | 47 | 52 | 52 | — |
| 4 | 52 | 49 | 52 | 52 | 3 |
| 5 | 52 | 49 | 52 | 52 | 28 |
| 6 | 52 | 49 | 52 | 52 | 28 |
| 7 | 52 | 49 | 52 | 52 | 28 |
| 8 | 52 | 49 | 52 | 52 | 28 |
| 9 | 52 | 49 | 52 | 52 | 28 |
| Total | 100% | 94% | 100% | 100% | 54% |

### Example 6

### CXCR4 Study

Replication Endpoint Concentration Assay

A TCID₅₀ of: 1000 for HIV-1_{Bal} (CCR5-using) and 10,000 for HIV-1_{Lai} CXCR4-using) was used to infect 10⁷ PHA-stimulated peripheral blood mononuclear cells in 24 well microtiter plates. The concentrations of recombinant, ultrapure immunokine used were 1-1000 μg/mL. All strains were tested in quadruplicate wells in three separate experiments. To correlate the replication endpoint concentration with a formal percent inhibitory concentration, we obtained that absolute p24 antigen content for each drug concentration. The concentration of drug that reduced the p24 antigen value of the control well by 50% (IC₅₀) was calculated using non-parametric regression analysis. Immunokine inhibited infection by HIV-1_{Bal} by 87% compared to untreated controls and inhibited infection by HIV-1_{Lai} by 96% compared to untreated controls with an IC₅₀ for CCR5-using isolates of 90 ηg/mL and an IC₅₀ of 10 μg/mL for CXCR4-using isolates of HIV-1 (see figure). Immunokine did not affect proliferation as measured by [³H]thymidine incorporation and was not cytotoxic as determined by the soluble formazan assay.

[image]

### Example 7

### Human Thymus Explant Culture

Human thymus removed for cardiac procedures from children ages 4.5 months to 11 years was grown in culture up to 7 days without loss of cells. A minimum of three replicate tissue pieces were harvested for each time point or condition normally yielding 3-6 million cells per/fragments. The tissue fragments were pretreated with 100 ηg/mL of immunokine for 1 hour at 37° C. The tissue fragments were washed in PBS, pH 7.4 and placed into sterile tubes containing 3000 TCID₅₀ of either HIV-1_{Bal} or HIV-1_{Lai}. The tissues were incubated at room temperature for 4 hours with gentle rocking. The tissue fragments were washed twice with PBS, pH 7.4 and transfered to 0.45 μm nucleopore filters (Millipore) atop gelfoam boats (Upjohn) saturated in media [(YSSL's, 1% human serum, 50 μg/ml streptomycin, 50 U/ml penicillin G, 1× MEM vitamin solution (GIBCO,BRL), 1× insulin/transferrin/sodium selenite media supplement (Sigma)], in six well plates with a maximum of 16 pieces per raft. The fragments were incubated at 37° C. with 5% CO₂ for up to 3 days. At day 3, 3-4 fragments were removed and processed for flow cytometry. Quantitative evaluation of T-cell precursor subsets was performed to determine if immunokine protected thymocytes from HIV-1 induced destruction in this in vivo model. As shown in the figure, 100 ηg/mL of immunokine protected CD4 and CD8 single positive T-cell precursors and CD8/CD4 dual positive T-cell precursors from the HIV-1 induced destruction seen in untreated controls.

[image]

What is claimed is:

1. A composition for preventing HIV infection of mammalian cells, the composition comprising an anti-immunodeficiency virus immunokine capable of binding to a cellular protein in a manner that prevents HIV infection of the cell.

2. A composition according to claim 1 wherein the immunodeficiency virus is selected from the group consisting of HIV-1,HIV-2 and SIV.

3. A composition according to claim 1 wherein the immunokine comprises an inactivated bioactive polypeptide.

4. A composition according to claim 3 wherein the inactivated bioactive polypeptide comprises a toxin selected from neurotoxins affecting the presynaptic neurojunction, toxins affecting postsynaptic neurojunction, and toxins affecting ion channels.

5. A composition according to claim 4 wherein the toxin comprises α-cobratoxin.

6. A composition according to claim 1 wherein the immunokine is adapted to bind one or more of a chemokine receptor protein, and a cellular cofactor for a cellular HIV receptor protein.

7. A composition according to claim 6 wherein the protein to which the immunokine of the invention binds is selected from the group consisting of CD4, CXCR4 and CCR5. consisting of CD4 and CXCR4 or CCR5

8. A composition according to claim 3 wherein the immunokine provides a substantially native toxin structure wherein one or more of the disulfide bridges are lacking by a method selected from the ozonation of native toxin, genetic engineering, and protein synthesis.

9. A composition according to claim 8 wherein ozonation is performed in a stoichiometric manner.

10. A composition according to claim 9 wherein the immunokine comprises inactivated alpha-cobratoxin in which the disulfide bridges are substantially lacking by ozonation of native alpha-cobratoxin.

11. A method of inhibiting infection of a cell by HIV comprising adding to the cell an anti-immunodeficiency virus immunokine capable of binding to a cellular protein on the cell, wherein upon binding of the immunokine to the cellular protein infection of the cell by HIV is inhibited.

12. A method of treating HIV infection in a human comprising administering to the human an anti-immunodeficiency virus immunokine capable of binding to a cellular protein on a cell, wherein upon binding of the immunokine to the cellular protein, infection of the cell by HIV is inhibited.

13. A method of preparing an anti-immunodeficiency virus immunokine capable of binding to a cellular protein on a cell, the method comprising the chemical, genetic and synthetic modification of native neurotoxins.

14. A method of identifying a target cell for immunodeficiency virus infection, the method comprising adding to a population of cells an anti-immunodeficiency virus immunokine capable of binding to a cellular protein on a cell, wherein binding of the immunokine to a cell in the population is an indication that the cell is an immunodeficiency virus target cell.

15. A method of identifying a candidate anti-immunodeficiency virus compound, the method comprising isolating a test compound capable of binding to an anti-immunodeficiency virus immunokine, which immunokine binds to a cellular protein, and assessing the ability of the test compound to inhibit infection of a cell by an immunodeficiency virus in an antiviral assay, wherein inhibition of infection of the cell by the immunodeficiency virus in the presence of the test compound is an indication that the test compound is an anti-immunodeficiency virus compound.
